(19) United States
(12) Patent Application Publication
(10) Pub. No.: US 2026/0165936 A1
(43) Pub. Date: Jun. 18, 2026

(54) BIO-BASED SURFACTANT COMPOSITION FOR REDUCTION OF MULTISPECIES BIOFILM FORMATION IN ORAL CAVITY

(71) Applicant: SKYLAB AG, Epalinges (CH)

(72) Inventors: Elena Yur'evna BELOUS, Moscow (RU); Alina Olegovna YAROVAYA, Moscow (RU)

(21) Appl. No.: 19/127,530

(22) PCT Filed: May 6, 2024
(86) PCT No.: PCT/EP2024/062382

§ 371 Date: May 6, 2025

(30) Foreign Application Priority Data
May 12, 2023 (EP) 23173010.2

(51) Int. Cl. A61K 8/60; A61K 8/44; A61Q 11/00

(52) U.S. Cl. CPC A61K 8/602; A61K 8/442; A61Q 11/00

(57) ABSTRACT

A composition for oral treatment, comprising rhamnolipid; and sodium lauroyl sarcosinate. The rhamnolipid is derived from bacterium of a genus Burkholderia, preferably from Pseudomonas spp., further preferably from Pseudomonas aeruginosa, further preferably rhamnolipid having the CAS number 4348-76-9. The composition is a mouthwash or a toothpaste, as a film, aerosol, suspension, solution, tincture, cream, paste, lotion, ointment, gel, powder, or granulate. The composition can be a medication used for prophylaxis or treatment of an oral disease, such as caries, periodontitis, gingivitis, tonsillitis, stomatitis, halitosis, dental calculus, or for treatment of an endodontic infection of a dental pulp, root or nerves of a living natural tooth.

### FIELD OF THE INVENTION

Removal of dental plaque is one of the main problems in oral care faced by humans. Dental plaque or dental biofilm is a highly-organized microbial community, which is attached to the solid surface of tooth. On the one hand the biofilm is a commensal part of the immune system, while on the other hand the uncontrolled activity of bacterial pathogens can cause a number of diseases including caries, periodontitis, endodontic infections, tonsillitis, alveolitis, etc. (Yaacob et al., 2014; Colombo et al., 2015; Valm, 2019).

### BACKGROUND OF THE INVENTION

The composition of dental plaque shows inter-individual variability and depends on a number of factors, however, there are some typical provisions characterizing dental plaque. Dental plaque is broadly classified as supragingival plaque, which is found at gingival margin or above the gingival margin, and subgingival plaque found below the gingival margin, between the tooth and gingival crevice (Haffajee et al., 2008).

There are several stages of the dental biofilm formation: 1) film formation prior to planktonic form attachment, 2) primary colonization, followed by proliferation of the attached microorganisms, 3) secondary colonization/coaggregation, 4) biofilm maturation (Souza et al., 2016; Digel et al., 2020). Gram positive cocci and bacilli predominate on the immediate tooth surface, while Gram negative bacilli and filamentous bacteria, as well as spirochetes, predominate on the surface of the formed biofilm (Haffajee et al., 2008). Besides bacteria, the fungi are also involved in the biofilm formation (Valm, 2019).

Mature biofilm is a multilayer structure made of heterogenous cell population surrounded by the extracellular matrix. The nutrients and the bacterial waste products circulate in numerous channels inside the matrix. Matrix is composed of exopolysaccharides, proteins and the macromolecules such as nucleoproteins and lipids. The matrix protects the bacteria from toxins, pH, osmotic pressure changes, ultraviolet radiation and dehydration (Shah et al., 2013).

The shift of equilibrium towards the pathogenic microbes is associated with high risk of development of diseases in oral cavity, such as caries and periodontitis. These are the most common diseases caused by modification of the composition of healthy microbiological society and by entry of the pathogenic microbes from the environment (Tanner et al., 2018; Valm, 2019). In this connection, it is necessary to maintain the healthy oral microbiota and timely removal of cariogenic biofilm. Gingivitis is another disease, which can be induced by the dental plaque. In the absence of appropriate oral care hygiene, the gingivitis can transform into chronic periodontitis (Kurhan and Kantarci, 2018; Valm, 2019).

In connection with the above, timely removal of dental plaque is essential, because uncontrolled dental plaque increase the risk of the oral cavity diseases. At the present time several types of control and prevention of dental plaque are available. These approaches can be classified as mechanical and chemical ones (Vyas et al., 2021). The medical professionals advise to perform the first type of cleaning, mechanical, yearly in the dentist's room (Axelsson and Odont, 1981), where several types of professional care are offered: mechanical treatment, air abrasion, ultrasonic treatment or laser treatment. Between professional cleaning sessions, the patient needs to employ prophylactic measures to prevent the dental calculus formation and to decelerate the dental plaque formation. Mechanical methods include the use of toothbrushes, dental flosses, gingival protectors, etc. (Vyas et al., 2021). Toothbrushes are the most commonly used among the above methods (Mandal et al., 2017). For efficacious prophylaxis it is necessary to use safe and efficient chemical agents as the ingredients of prophylactic oral care products.

Anti-plaque agents are subdivided into three generations based on various approaches. The first-generation agents, such as antibiotics, phenols and other compounds, are compounds decreasing the dental plaque level by 20-50%. The second-generation approach is based on the use of chlorohexidine. Despite efficient (70-90%) removal of dental plaque, this approach has a number of side effects, including: hypogeusia, discomfort caused by butter taste, burning sensation, dryness and tenderness of mucosa, and epithelium desquamation. The third-generation agents block the attachment of microbes to the tooth surface (Vyas et al., 2021).

Since many anti-plaque agents are low-efficacious or lead to injury of mucosa, there is an unsatisfied need to develop new agents, which would be safe for the soft tissues of oral cavity and efficiently control plaque formation. This makes very relevant the inclusion of such group of compounds as surfactants into the formulations of oral care products, especially those that are milder and more environmentally compliant. The inventors of the present invention found that composition containing biosurfactant rhamnolipid and sodium lauroyl sarcosinate efficiently prevent the formation of the dental plaque.

Surfactants are chemically surface-active compounds, which are used in household chemicals, cosmetics, and oral care products. Surfactants are a key component of the toothpastes, where they are responsible for the foaming activity. Surfactants contribute to uniform distribution of the toothpaste in the oral cavity and to the cleaning process, in particular, to removal of debris, microbial biofilm, and the dental plaque from the tooth surface. Another surfactant function is the formation of micelles containing flavor fillers of the toothpaste (Lindenmüller et al., 2011; Aspinall et al., 2021).

Despite beneficial surfactants' aspects and the important role thereof in toothpaste compositions, many synthetic surfactants can case irritation and contamination of the environment subject to entry to the environment (Akbari et al., 2018; Naughton et al., 2019; Aspinall et al., 2021). For instance, large amounts of surfactant can cause the irritation of mucosa, while insufficient amount exhibits considerably lower activity (Aspinall et al., 2021). The surfactants can enter the environment when used in production of various raw materials—in pharmaceutical industry, food and feed industries, agriculture and oil industry.

Safety concerns associated with environmental disadvantages led to establishment of certain standards and social pressure aimed to transition to more biodegradable agents and sustainable production of the raw materials (Naughton et al., 2019). This contributed to reduction of the synthetic surfactant fraction in the toothpastes up to complete absence thereof. However, surfactant-free toothpastes lack useful properties, which characterize the surfactant-based pastes. The solution of choice is to produce the pastes containing biosurfactants or bio-synthetic surfactant compositions to minimize the harmful effects both on oral mucosa and on the environment.

Biosurfactants exhibit a number of advantages compared to synthetic surfactants, such as low toxicity, bioavailability, biodegradation, high foaming activity, environmental friendliness, low cost due to availability of natural raw materials, and a wide range of pH and salinity, in which the surfactant activity is maintained (Fracchia et al., 2015; Akbari et al., 2021). Besides the use in the oral care products, the biosurfactants are used in beauty products, pharmaceutics, food industry, agriculture, and textile dyeing. These multifunctional agents exhibit the stabilizing, antimicrobial, moisturizing, emulsifying, and anti-adhesive activities (Banat et al., 2000; Fracchia et al., 2014).

Natural surfactants are produced from the natural raw materials, and some are produced with the use of microorganisms. The production of biosurfactants exhibiting antibacterial, fungicidal, and antiviral properties by bacteria is of great interest. The bacteria of genera Pseudomonas and Bacillus have the greatest potential, but taking into account the pathogenicity of these genera, yeast and non-pathogenic bacteria are preferable (Makkar and Cameotra, 2002; Naughton et al., 2019).

The surfactants can be subdivided into the agents produced by chemical synthesis and those obtained from the natural raw materials, i.e., biosurfactants. The latter are classified based on the molecular weight, their origin, and composition. High-molecular biosurfactants include lipopolysaccharide-protein complexes, polysaccharide-protein-fatty acid or lipoprotein complexes, while glycolipids, lipopeptides, and phospholipids are the low-molecular biosurfactants. Low-molecular biosurfactants more efficiently decrease the surface tension and interfacial tension, while high-molecular biosurfactants are more potent emulsifiers (Banat et al., 2010).

The glycolipids having the greatest therapeutic potential are of primary interest among the studied biosurfactants. In the glycolipid molecule, carbohydrate fragments are linked to fatty acids of varying length. This group includes trehalolipids, cellobiose lipids, mannosyl erythritol lipids, rhamnolipids, and sophorolipids (Mnif and Ghribi, 2016; Marchant and Banat, 2012; Adu et al., 2020). The glycolipids produced by the microorganisms are exemplified by the rhamnolipids produced by Pseudomonas spp., sophorolipids and mannosyl erythritol lipids produced by Candida spp., trehalose lipids produced by Mycobacterium and Rhodococcus spp (Santos et al., 2016; Vecino et al., 2017; Peyrat et al., 2019).

Rhamnolipids are mainly synthesized by Pseudomonas aeruginosa and the other species of genus Burkholderia. The rhamnolipids are classified based on the number of the deoxysaccharide rhamnose residues, which are attached by O-glycoside bond to one or two fatty acid chains. The latter vary in length from 8 to 14 carbon atoms, and 10-carbon rhamnolipids are the most prevalent. One rhamnose gives rise to monorhamnolipid, two rhamnoses give rise to dirhamnolipid (Abdel-Mawgoud et al., 2010; Elshikh et al., 2017).

The rhamnolipids have the greatest potential for expansion at the market of beauty products and household chemicals compared to the other biosurfactants (Müller et al., 2012). This can be explained by the distinguishing properties of rhamnolipids, i.e., the combination of ability to decrease the surface tension, low critical concentration of micelle formation and high washing and foaming activities, antimicrobial properties, gradual substrate sorption, and excellent capacity for molecular complex and liquid crystal formation. Furthermore, the rhamnolipids maintain their properties in a wide temperature, pH, and salinity ranges (Moussa et al., 2014; Sharma, 2016; Kumar and Das, 2018).

Some of synthetic surfactants are non-toxic despite artificial origin and can be used in biodegradable compositions. One of them is sodium lauroyl sarcosinate (SLS). This moderate biodegradable anionic surfactant is made up of a fatty acid and an amino acid sarcosine (Tackie-Otoo et al., 2022). The studies of N-acyl sarcosine derivatives showed that SLS is not a potentially toxic chemical, because the derivatives thereof readily decompose to amino acids and fatty acids (Lanigan, 2001; Tripathy et al., 2018).

The use of rhamnolipids and SLS in the field of oral care products, in particular, for inhibition of the microbial growth, as individual agent and as ingredient of various compositions is reported in the literature.

Elshikh et al. studied the antimicrobial activity of rhamnolipids isolated from non-pathogenic Burkholderia thailandensis, against some microorganisms, which are found in the oral cavity: Streptococcus mutans, Streptococcus oralis, Streptococcus sanguinis, Neisseria mucosa and Actinomyces naeslundii. These microorganisms, alongside with some other species, are responsible for biofilm formation on teeth surfaces, which can lead to dental calculus deposition and caries (Kolenbrander et al., 2010). The authors found that rhamnolipids (both alone and in combination with the other antimicrobial agents, such as tetracycline, chlorhexidine, sodium lauroyl sulphate and ciprofloxacin) prevent the biofilm formation and also destroy existing biofilms (in the absence of any other components). The researchers showed high penetrating capacity of rhamnolipids into the bacterial cells (Elshikh et al., 2017).

Yamasaki et al. also demonstrated, in particular, in relation to A. actinomycetemcomitans Y4, S. mutans UA159 and S. sanguinis ATCC10556, that rhamnolipids inhibit growth and formation of the biofilms in the oral cavity (Yamasaki et al., 2020).

Mynenivenkatasatya et al. studied the efficacy of the toothpaste containing SLS and also the stabilized chlorine dioxide and sodium fluoride, in particular, the effect on formation of microbial biofilm. The authors showed high antimicrobial activity of the examiner toothpaste compared to control (Mynenivenkatasatya et al., 2020).

The inventors of the present invention showed the unique synergistic action of rhamnolipid when used in combination with SLS. The composition showed high activity against formation of microbial biofilms compared to activities of individual components, i.e., of biosurfactant and SLS. Such unique activity can be associated with the distinguishing properties of individual components, which potentiates due to concomitant use. For instance, SLS, a “green” synthetic surfactant, is also an inhibitor of hexokinase, which is a first enzyme in a metabolic pathway of glucose and some other sugars (Carbon et al., 1995; Fosdick, 1956; Bajani et al., 2018). In this view, the sugars can be metabolized to a lesser extent by bacteria, in particular by Streptococcus mutans, thus providing additional teeth protection from the plaque formation and caries development (Forssten et al., 2010). When used with rhamnolipids, which are also efficient surfactants and exhibit antimicrobial activity, in particular, disturb the growth of fungi, bacteria, and other microorganisms, the composition provides and efficacious control over the dental plaque (Abalos et al., 2001; Benincasa et al., 2004; Lourith and Kanlayavattanakul, 2009). Previously the use of rhamnolipids in cosmetics was described, in particular, in the oral care products. Nevertheless, the combination and the unique properties thereof are innovative in nature.

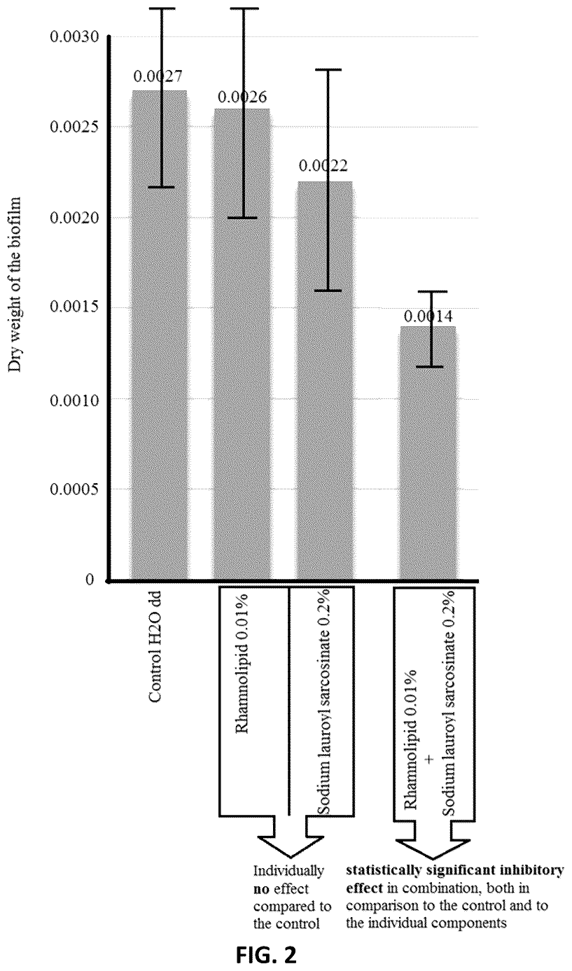

US 2019307657 A1 (EVONIK DEGUSSA GMBH [DE]) published on Oct. 10, 2019, discloses the use of the oral care composition containing at least one biosurfactant and at least one source of fluorine ions. Patented biosurfactants include rhamnolipids, sophorolipids, the lipids containing a glucose residue, trehalolipids, and cellulose lipids. The composition may also comprise other surfactant of non-biological origin. The authors of the patent highlighted the advantages of the composition, including low level of insoluble fluorine complexes, decreased tooth coloration and deodorization of oral cavity, better taste of the oral care products and better fluorine deposition/retention on the surfaces together with decrease in dental plaque. Importantly, while US 2019/0307657A1 claims that “A further advantage of the present invention is that the compositions reduce dental plaque,” in paragraph [0018], it does not provide any evidence, let alone any examples or data supporting that claim. This is in sharp contrast to the present invention. The example of the present application shows clearly in terms of hard data what is defined in the independent claims of the appended claim set, namely the successfully treatment of plaque and/or biofilm or for prevention of a plaque and/or biofilm formation. It is important to understand that the composition of the present invention comprises two essential core components, namely rhamnolipid and sodium lauroyl sarcosinate, which only in combination, i.e., when acting in concert, provide for the (statistically significant) synergistic effect of biofilm inhibition, while the individual components alone, i.e., without its partner, do not provide for any biofilm inhibitory effect compared to the control, as can be clearly seen from the data shown in FIG. 2 of the present application. US 2019/0307657 A1 does not provide any data on biofilm inhibition, neither for rhamnolipid and sodium lauroyl sarcosinate alone nor in combination. Furthermore, US 2019/0307657 A1 does not teach or suggest the importance of the combined application of rhamnolipid and sodium lauroyl sarcosinate to arrive on a synergistic inhibitory effect on a biofilm. As such, any suggestion that one would arrive at the claimed invention as defined in the claims in an obvious manner starting from US 2019/0307657 A1 necessarily relies on impermissible hindsight reconstruction.

The same applies to published EP 3 875 1001 A1, which relates to a composition comprising rhamnolipids and sodium lauroyl sarcosinate for use as a medicament or a cosmetic product for regeneration of the skin, mucous membrane and cutaneous annexes, wound treatment or as a cosmetic ingredient for protection, maintenance and natural balance of the mucous membrane and cutaneous annexes, without any relevance to the gist of the present application as defined in the claims, namely the use of the combination of rhamnolipid and sodium lauroyl sarcosinate for biofilm inhibition.

Patent Application WO 2019133313 A1 published on Jul. 4, 2010 (LOCUS IP CO LLC [US]) discloses the use of the composition for amelioration and/or maintenance of the oral health. The composition contains one or more purified biosurfactants and/or derivative thereof, and optionally mat contain one or more carriers, additives and/or adjuvants. The claimed biosurfactants include glycolipids, such as rhamnolipids, sophorolipids, trehalilipids and mannosyl erythritol lipids. While the Applicant of WO 2019133313 A1 indicates the use of the composition as the product for teeth whitening, removal and/or prevention of dental plaque, biofilm or dental calculus and decrease in the population of unfavorable microorganisms in the oral cavity, etc., none of the claimed uses was technically supported. Instead, the authors provided the examples of 1) lipopeptide production by Bacillus spp., 2) fermentation by Starmerella bombicola for biosurfactant production, 3) the variant of the tooth paste composition.

US 20210169757 A1 (COLGATE PALMOLIVE CO [US]) discloses the composition for the oral care products, which maintains viscosity, and the methods of use thereof. The ingredients of the composition include 1) basic amino acid, 2) the source of zinc ions, 3) a surfactant system, which comprises one or more alkyl glycosides, acyl glutamates, glycolipids or combinations thereof. According to the authors, the properties of the composition include decrease in acidogenic bacteria population, lesser microbial biofilm formation in the oral cavity, and also decrease or inhibition of the plaque formation. Despite the claimed properties, the Applicant of US 20210169757 A1 demonstrated only the maintenance of viscosity, taste, and foaming activity of the toothpaste. Furthermore, for rhamnolipid-containing pastes, only the first two characteristics are demonstrated.

U.S. Pat. No. 7,985,722B2 (AURORA ADVANCED BEAUTY LABS [US]) published on Jul. 26, 2011, discloses the use of rhamnolipid-based compositions for cleaning, disinfection, and deodorization of living areas and working rooms. The composition of the invention comprises 0.01% to 99.9% (preferably, 0.01% to 70%) of rhamnolipids, and carrier is the rest. The authors report that such compositions additionally form the biofilm, which prevents proliferation of bacteria and fungi. The patent text, alongside with the other numerous optional uses, indicates the use of the composition as an ingredient of the toothpastes. However, in the examples of uses pertinent to the oral care products the authors described only the use of the composition for antimicrobial and fungicidal coating on the toothbrushes, where the rhamnolipid antimicrobial properties and the surface tension maintained for a week. Nevertheless, since no experiments demonstrating the claimed rhamnolipid properties of the other oral care products were made, the activity thereof is not proved.

Lauroyl sarcosinate (LS) has been patented as an ingredient of many compositions, though these compositions do not include rhamnolipids. For instance, US 2022023180 A1 (COLGATE PALMOLIVE CO [US]) published on Jan. 27, 2022, provides the composition containing lauroyl sarcosinate and betaine for use in the oral care products. The authors of the publication described several potential compositions and the foaming activities thereof, however the efficacy against the microbial biofilm was not demonstrated.

Thus, the authors of the present invention developed the innovative composition containing the rhamnolipids and LS. This composition unexpectedly showed high synergetic efficacy in decrease of the dental plaque weight compared to the individual components and can be used as the ingredient of cosmetic products for efficient cleaning of teeth and oral cavity.

### DISCLOSURE OF THE INVENTION

The invention is set out in the appended claims. The invention is related to the composition containing the following two components (A) and (B): (A) rhamnolipid; and (B) sodium lauroyl sarcosinate.

The rhamnolipid can be produced from genus Burkholderia bacteria, preferably, from Pseudomonas spp., more preferably from Pseudomonas aeruginosa, and even more preferably the rhamnolipid is identified by CAS 4348-76-9.

The level of component (A) in the composition may be selected from the following ranges, wt %: preferably 0.004-5.000; more preferably 0.006-4.000; still more preferably 0.008-3.000; still more preferably 0.010-2.000; still more preferably 0.011-1.900; still more preferably 0.012-1.800; still more preferably 0.013-1.700; still more preferably 0.014-1.600; still more preferably 0.015-1.500; still more preferably 0.015-1.400; still more preferably 0.015-1.300; still more preferably 0.015-1.200; still more preferably 0.015-1.100 or 0.015-1.000. For example, given the above ranges, the amount of component (A) in the composition may be, in, wt %: 0.004; preferably 0.005; still more preferably 0.006; still more preferably 0.007; still more preferably 0.008; still more preferably 0.009; still more preferably 0.01; still more preferably 0.015; still more preferably 0.02; still more preferably 0.025; still more preferably 0.03; still more preferably 0.035; still more preferably 0.04; still more preferably 0.045; still more preferably 0.05; still more preferably 0.055; still more preferably 0.06; still more preferably 0.065; still more preferably 0.07; still more preferably 0.075; still more preferably 0.08; still more preferably 0.085; and so on, with 0.09; 0.095; 0.1; 0.15; 0.2; 0.25; 0.3; 0.35; 0.4; 0.45; 0.5; 0.55; 0.6; 0.65; 0.7; 0.75; 0.8; 0.85; 0.9; 0.95; 1; 1.05; 1.1; 1.15; 1.2; 1.25; 1.3; 1.35; 1.4; 1.45; 1.5; 1.55; 1.6; 1.65; 1.7; 1.75; 1.8; 1.85; 1.9; 1.95; 2.00; 2.05; 2.1; 2.15; 2.2; 2.25; 2.3; 2.35; 2.4; 2.45; 2.5; 2.55; 2.6; 2.65; 2.7; 2.75; 2.8; 2.85; 2.9; 2.95; 3.00; 3.05; 3.1; 3.15; 3.2; 3.25; 3.3; 3.35; 3.4; 3.45; 3.5; 3.55; 3.6; 3.65; 3.7; 3.75; 3.8; 3.85; 3.9; 3.95; 4.00; 4.05; 4.1; 4.15; 4.2; 4.25; 4.3; 4.35; 4.4; 4.45; 4.5; 4.55; 4.6; 4.65; 4.7; 4.75; 4.8; 4.85; 4.9; 4.95; or 5.00. Furthermore, the level of component (A) in the composition may be selected from the following ranges, wt %: 0.06-0.20; 0.07-0.19; 0.08-0.18; 0.09-0.17; 0.1-0.16; 0.11-0.15; 0.12-0.14; or 0.13-0.14. For example, the level of component (A) in the composition is selected from the following, wt %: preferably 0.06; more preferably 0.07; and so on, with 0.08; 0.09; 0.1; 0.11; 0.12; 0.13; 0.14; 0.15; 0.16; 0.17; 0.18; 0.19; or 0.2.

The level of component (B) in the composition may be selected from the following ranges, wt %: preferably 0.05-22.00; more preferably 0.10-11.00; or 0.20-5.50. For example, the level of component (B) in the composition is selected from the following, wt %: preferably 0.05; more preferably 0.06; still more preferably 0.07, and so on: 0.08; 0.09; 0.1; 0.15; 0.2; 0.25; 0.3; 0.35; 0.4; 0.45; 0.5; 0.55; 0.6; 0.65; 0.7; 0.75; 0.8; 0.85; 0.9; 0.95; 1; 1.5; 2; 2.5; 3; 3.5; 4; 4.5; 5; 5.5; 6; 6.5; 7; 7.5; 8; 8.5; 9; 9.5; 10; 10.5; 11; 11.5; 12; 12.5; 13; 13.5; 14; 14.5; 15; 15.5; 16; 16.5; 17; 17.5; 18; 18.5; 19; 19.5; 20; 20.5; 21; 21.5; or 22. Furthermore, the level of component (B) in the composition may be selected from the following ranges, wt %:1.0-2.0; 1.1-1.9; 1.2-1.8; 1.3-1.7; 1.4-1.6; or 1.5-1.6. For example, the level of component (B) in the composition is selected from the following, wt %: preferably 1.0; more preferably 1.1; and so on with 1.2; 1.3; 1.4; 1.5; 1.6; 1.7; 1.8; 1.9; or 2.0.

The weight ratio of sodium lauroyl sarcosinate and rhamnolipid in the combination may be as follows: 0.07:2.0 to 0.004:0.2, respectively. In other words, within the range the weight content can be as follows: preferably 0.004; more preferably 0.005; and so on with 0.006; 0.007; 0.008; 0.009; 0.01; 0.015; 0.02; 0.025; 0.03; 0.035; 0.04; 0.045; 0.05; 0.055; 0.06; 0.065; 0.07 and 0.2; 0.25; 0.3; 0.35; 0.4; 0.45; 0.5; 0.55; 0.6; 0.65; 0.7; 0.75; 0.8; 0.85; 0.9; 0.95; 1; 1.5; 2 respectively.

The composition may be used for oral care.

The oral care composition may be a mouthwash, and/or a toothpaste.

In one example, the composition may contain, in weight percent, rhamnolipid 0.004-1.0%; sodium lauroyl sarcosinate 0.05-5.50%; purified water 5-90%; preservative 0.01-2%; humectant 5-40%; solubiliser 0.01-10%; and sweetener 0.001-5%, where rhamnolipid:sodium lauroyl sarcosinate wt % ratio is between 0.07:2.0 and 0.004:0.2. To make a toothpaste using this composition, it may contain, for example, in weight percent, rhamnolipid 0.004-1.0%; sodium lauroyl sarcosinate 0.05-5.50%; purified water 5-90%; preservative 0.01-2%; humectant 5-40%; solubiliser 0.01-10%; abrasives (silicon dioxide or/and calcium carbonate or/and calcium phosphates or/and charcoal or/and zeolites or/and aluminum oxide) 0.01-30%; thickener (carboxymethylcellulose or/and xanthan gum or/and Carbomers or/and Sodium alginate or/and Carrageenan or/and hydroxypropyl methylcellulose or/and Hydroxyethylcellulose) 0.1-5%; and sweetener 0.001-5%, where rhamnolipid:sodium lauroyl sarcosinate wt % ratio is between 0.07:2.0 and 0.004:0.2. To make a mouthwash using this composition, it may contain, for example, in weight percent, rhamnolipid 0.004-1.0%; sodium lauroyl sarcosinate 0.05-5.50%; purified water 5-90%; preservative 0.01-2%; humectant 5-40%; solubiliser 0.01-10%; flavoring agent—0.1-3%; and sweetener 0.001-5%, where rhamnolipid:sodium lauroyl sarcosinate wt % ratio is between 0.07:2.0 and 0.004:0.2.

The composition can be an oral care composition, wherein the oral care composition is preferably a formulation selected from the following: a film, an aerosol, a suspension, a solution, a tincture a cream, a lotion, an ointment, a gel, a powder, or a granulate.

The composition can be used for nonmedical and/or aesthetic purpose.

In a further aspect the invention is related to the use of composition of the invention for prevention or treatment of forming dental plaque and/or biofilm in the mouth and/or on teeth.

The dental plaque and/or biofilm may be derived from the oral flora in the mouth, wherein the oral flora can be a microorganism-based oral mouth flora, wherein further preferably the microorganisms may be selected from the group comprising Streptococcus mutans, Streptococcus oralis, Streptococcus sanguinis, Neisseria mucosa and Actinomyces naeslundii.

In a further aspect the invention is related to a pharmaceutical composition or medicament containing the composition of the invention.

In a further aspect the invention is related to the composition of the invention to be used as a pharmaceutical composition or medicament, wherein the medicament is preferably formulated to be used in the oral cavity of the subject.

In a further aspect, the medicament or a composition of the invention can be used in treatment of the subject having at least one of the following diseases or symptoms: gingivitis, stomatitis, offensive breath, sensitive teeth, caries or dental calculus.

### EXAMPLES

### Example 1

The aqueous solutions of sodium lauroyl sarcosinate, biosurfactant rhamnolipid and the mixture thereof at various concentrations were tested for inhibition of biofilm formation on the surface of rough glass rods. The test sample of the raw material contained sodium lauroyl sarcosinate at the level of 35%. The test sample of the raw material contained rhamnolipid at the level of 40%.

Sterile water was used as a negative control, and the mouthwash Corsodyl, containing 0.2% chlorhexidine was used as a positive control. Dry biofilm mass was weighted to determine the amount of formed dental plaque.

The dental plaque was grown for 3 days by immersion of rough glass rods into the fresh human saliva containing 0.1% saccharose. On Day 2 and Day 3 the growth of biofilm was additionally stimulated by addition of nutritive broth containing tryptone-soya broth (TSB), saliva and saccharose. On Day 1 one treatment of the glass rods with the test component solutions, and on Days 2 and 3 two treatments of the glass rods with the test component solutions were made. At the end of Day 3 the biofilm was collected from the glass rods, and the plaque amount by dry weight (g) was determined. Lesser weight indicated higher efficacy against the dental plaque.

### Experimental Procedure

Sterilized rods were fixed in the holders in the microbiological cabinet. Since that moment the rods and the holders were used as the integral units.

On Day 1 the sterile glass rods were pre-treated for 2 minutes with the test component solutions taken in the amount of 5 mL. Then the glass rods were removed, washed in 5 mL of the sterile distilled water and immersed into 5 mL of saliva containing 0.1% saccharose. The tubes were placed into the rack and then into the incubator (t=37° C.) and on shaker (250 rpm) for about 18 hours.

On Days 2 and 3 in the morning the rods were treated with the test component solutions, then placed into nutritive broth and returned into the incubator and on shaker. After 6 h incubation in the broth the rods were treated with the test component solution once again and transferred into the saliva with 0.1% glucose, and then returned into the incubator for 18 hours.

On Day 4 the tubes with the rods were removed from the incubator and used for determination of the biofilm dry weight. For this purpose, the rods were dried and then weighted on analytical balance with the accuracy of 4 decimal places. The biofilm was removed after the rehydration, and the rods were weighted once again. The biofilm dry weight was determined from the equation:

$$Biofilm weight ( g ) = Weight of the road covered with biofilm ( g ) - Rod weight ( g )$$

### Experimental Results

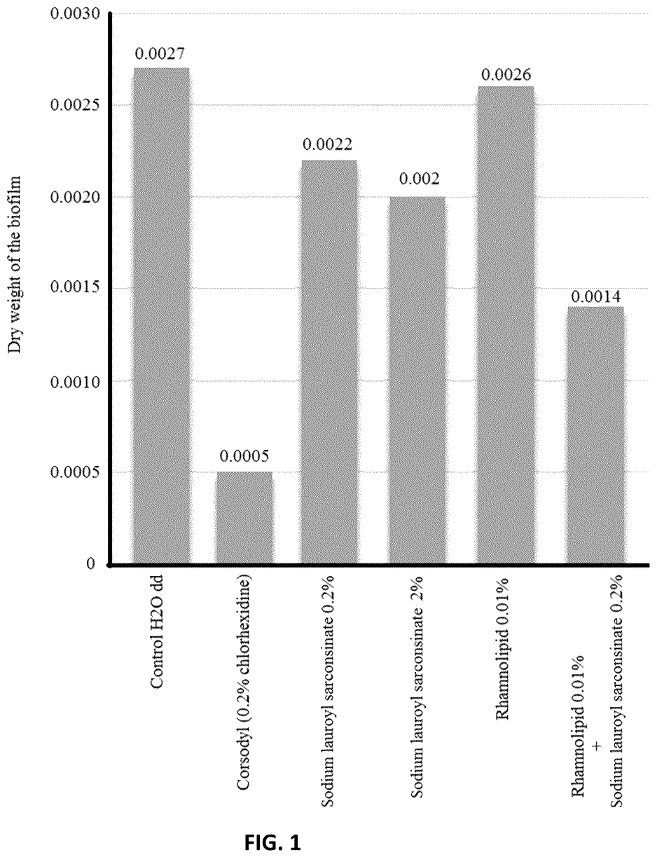

The results obtained for the anti-biofilm activity of the tested components are given in Table 1 and FIG. 1, wherein FIG. 2 summarizes the core of the invention, i.e., the statistically significant synergistic inhibitory effect of the claimed composition comprising the combination of rhamnolipid and sodium lauroyl sarcosinate compared to the control and the components individually being on the control level.

TABLE 1
Dry weight of biofilm (g).

| | Average biofilm | Standard |
|---|---|---|
| Component | weight (g) | deviation |
| Biosurfactant rhamnolipid 0.01% | 0.0026 | 0.0006 |
| Sodium lauroyl sarcosinate 0.2% | 0.0022 | 0.0006 |
| Sodium lauroyl sarcosinate 0.2% + | 0.0014 | 0.0002 |
| Biosurfactant rhamnolipid 0.01% | | |
| Sodium lauroyl sarcosinate 2% | 0.0020 | 0.0004 |
| Distilled water | 0.0027 | 0.0005 |
| Corsodyl (0.2% chlorhexidine) | 0.0005 | 0.0002 |

As is evident from Table 1, the combination of sodium lauroyl sarcosinate 0.2% and rhamnolipid 0.01% provides significant decrease in the dental plaque formation. The dental plaque weights in the event of the rod treatment with 0.2 and 2% aqueous sodium lauroyl sarcosinate solutions are virtually identical thus indicating the absence of obvious dose-dependent effect: 10-fold increase in concentration of this component does not lead to better efficacy in terms of decrease of the dental plaque formation. However, the addition of just 0.01% rhamnolipid solution to 0.2 sodium lauroyl sarcosinate solution results in considerably lower weight of dental biofilm, thus demonstrating the obvious synergistic effect of the components in tested concentrations.

It should also be appreciated that various modifications, adaptations and alternative embodiments thereof may be made within the scope and spirit of the present invention. The invention is further defined by the following claims.

### REFERENCES (INCORPORATED HEREIN BY REFERENCE IN THEIR ENTIRETY)

1. Abalos A. et al. Physicochemical and antimicrobial properties of new rhamnolipids produced by Pseudomonas aeruginosa AT10 from soybean oil refinery wastes //Langmuir.—2001.—T. 17.—No. 5.—C. 1367-1371.
2. Abdel-Mawgoud A. M., Lépine F., Déziel E. Rhamnolipids: diversity of structures, microbial origins and roles/Applied microbiology and biotechnology.—2010.—T. 86.—No. 5.—C. 1323-1336.
3. Adu S. A. et al. Microbial biosurfactants in cosmetic and personal skincare pharmaceutical formulations //Pharmaceutics.—2020.—T. 12.—No. 11.—C. 1099.
4. Akbari S. et al. Biosurfactants—a new frontier for social and environmental safety: a mini review/Biotechnology Research and Innovation.—2018.—T. 2.—No. 1.—C. 81-90.
5. Aspinall S. R., Parker J. K., Khutoryanskiy V. V. Oral care product formulations, properties and challenges //Colloids and Surfaces B: Biointerfaces.—2021.—T. 200.—C. 111567.
6. Axelsson P., Odont D. Concept and practice of plaque-control //Pediatr Dent.—1981.—T. 3.—No. Sp. Issue.—C. 101-113.
7. Bajani D., Gharai D., Dey J. A comparison of the self-assembly behaviour of sodium N-lauroyl sarcosinate and sodium N-lauroyl glycinate surfactants in aqueous and aqueo-organic media //Journal of colloid and interface science.—2018.—T. 529.—C. 314-324.
8. Banat I. M., Makkar R. S., Cameotra S. S. Potential commercial applications of microbial surfactants //Applied microbiology and biotechnology.—2000.—T. 53.—No. 5.—C. 495-508.
9. Benincasa M. et al. Chemical structure, surface properties and biological activities of the biosurfactant produced by Pseudomonas aeruginosa LBI from soapstock //Antonie Van Leeuwenhoek.—2004.—T. 85.—No. 1.—C. 1-8.
10. Carbon J. A. et al. The inhibition of anaerobic glycolysis by sodium N-lauroyl sarcosinate //Archives of Biochemistry and Biophysics.—1955.—T. 55.—No. 2.—C. 356-364.
11. Elshikh M. et al. Rhamnolipids and lactonic sophorolipids: natural antimicrobial surfactants for oral hygiene //Journal of applied microbiology.—2017.—T. 123.—No. 5.—C. 1111-1123.
12. Forssten S. D., Björklund M., Ouwehand A. C. Streptococcus mutans, caries and simulation models //Nutrients.—2010.—T. 2.—No. 3.—C. 290-298.
13. Fosdick L. S. Clinical experiment on the use of sodium N-lauroyl sarcosinate in the control of dental caries //Science.—1956.—T. 123.—No. 3205.—C. 988-989.
14. Fracchia L. et al. Industrial applications of biosurfactants //Biosurfactants: production and utilization-processes, technologies, and economics.—2014.—C. 245-260.
15. Fracchia L. et al. Potential therapeutic applications of microbial surface-active compounds //AIMS Bioengineering.—2015.—T. 2.—No. 3.—C. 144-162.
16. Kolenbrander P. E. et al. Oral multispecies biofilm development and the key role of cell-cell distance //Nature Reviews Microbiology.—2010.—T. 8.—No. 7.—C. 471-480.
17. Kumar R., Das A. J. Future Prospects and Scenario of Rhamnolipids //Rhamnolipid Biosurfactant.-Springer, Singapore, 2018.—C. 137-141.
18. Kumar R., Das A. J. Industrial Applications of Rhamnolipid: An Innovative Green Technology for Industry //Rhamnolipid Biosurfactant.-Springer, Singapore, 2018.—C. 65-77.
19. Lanigan R. S. Final report on the safety assessment of Cocoyl Sarcosine, Lauroyl Sarcosine, Myristoyl Sarcosine, Oleoyl Sarcosine, Stearoyl Sarcosine, Sodium Cocoyl Sarcosinate, Sodium Lauroyl Sarcosinate, Sodium Myristoyl Sarcosinate, Ammonium Cocoyl Sarcosinate, and Ammonium Lauroyl Sarcosinate //International journal of toxicology.—2001.—T. 20.—C. 1-14.
20. Lindenmüller I. H., Lambrecht J. T. Oral care //Topical applications and the mucosa.—2011.—T. 40.—C. 107-115.
21. Lourith N., Kanlayavattanakul M. Natural surfactants used in cosmetics: glycolipids //International journal of cosmetic science.—2009.—T. 31.—No. 4.—C. 255-261.
22. Makkar R., Cameotra S. An update on the use of unconventional substrates for biosurfactant production and their new applications //Applied microbiology and biotechnology.—2002.—T. 58.—No. 4.—C. 428-434.
23. Mandal A. et al. New dimensions in mechanical plaque control: An overview //Indian Journal of Dental Sciences.—2017.—T. 9.—No. 2.—C. 133.
24. Marchant R., Banat I. M. Biosurfactants: a sustainable replacement for chemical surfactants?//Biotechnology letters.—2012.—T. 34.—No. 9.—C. 1597-1605.
25. Mnif I., Ghribi D. Glycolipid biosurfactants: main properties and potential applications in agriculture and food industry //Journal of the Science of Food and Agriculture.—2016.—T. 96.—No. 13.—C. 4310-4320.
26. Moussa T. A. A., Mohamed M. S., Samak N. Production and characterization of di-rhamnolipid produced by Pseudomonas aeruginosa TMN //Brazilian Journal of Chemical Engineering.—2014.—T. 31.—C. 867-880.
27. Müller M. M. et al. Rhamnolipids next generation surfactants?//Journal of biotechnology.—2012.—T. 162.—No. 4.—C. 366-380.
28. Mynenivenkatasatya S. R. et al. Effectiveness of a novel dentifrice containing stabilized chlorine dioxide, sarkosyl, and sodium fluoride //Dentistry journal.—2020.—T. 8.—No. 4.—C. 122.
29. Naughton P. J. et al. Microbial biosurfactants: current trends and applications in agricultural and biomedical industries //Journal of applied microbiology.—2019.—T. 127.—No. 1.—C. 12-28.
30. Peyrat L. A. et al. Terrestrial microorganisms: cell factories of bioactive molecules with skin protecting applications //Molecules.—2019.—T. 24.—No. 9.—C. 1836.
31. Santos D. K. F. et al. Biosurfactants: multifunctional biomolecules of the 21st century //International journal of molecular sciences.—2016.—T. 17.—No. 3.—C. 401.
32. Sharma D. Biosurfactants in food.—Cham, Switzerland: Springer International Publishing, 2016.
33. Tackie-Otoo B. N. et al. Experimental investigation of N-lauroyl sarcosine and N-lauroyl-L-glutamic acid as green surfactants for enhanced oil recovery application //Journal of Molecular Liquids.—2022.—T. 362.—C. 119738.
34. Tripathy D. B. et al. Synthesis, chemistry, physicochemical properties and industrial applications of amino acid surfactants: A review //Comptes Rendus Chimie.—2018.—T. 21.—No. 2.—C. 112-130.
35. Vecino X. et al. Biosurfactants in cosmetic formulations: trends and challenges //Critical reviews in biotechnology.—2017.—T. 37.—No. 7.—C. 911-923.
36. Vyas T. et al. Chemical plaque control-A brief review //Journal of Family Medicine and Primary Care.—2021.—T. 10.—No. 4.—C. 1562.
37. Yamasaki R. et al. Rhamnolipids and surfactin inhibit the growth or formation of oral bacterial biofilm //BMC microbiology.—2020.—T. 20.—No. 1.—C. 1-11.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. An oral care composition, comprising:
rhamnolipid 0.004-1.0%;
sodium lauroyl sarcosinate 0.05-5.50%;
purified water 5-90%;
preservative 0.01-2%;
humectant 5-40%;
solubiliser 0.01-10%; and
sweetener 0.001-5%,
wherein rhamnolipid:sodium lauroyl sarcosinate wt % ratio is between 0.07:2.0 and 0.004:0.2.

17. The oral care composition of claim 16, wherein the preservative is sodium benzoate or benzoic acid or biosol (O-cymen-5-ol) or potassium sorbate or bisabolol or saponins or Natural Extracts and Blends (Japanese Honeysuckle Extract) or parabens or Phenoxyethanol or sorbic acid or Ethylhexylglycerin or Caprylyl Glycol or any combination thereof.

18. The oral care composition of claim 16, wherein the humectant is sarbitol or glycerol or propylene glycol or polyethylene glycol or any combination thereof.

19. The oral care composition of claim 16, wherein the solubiliser is Polyglyceryl-4 Laurate, Sebacate or Polyglyceryl-6 Caprylate, Caprate or PEG-40 Hydrogenated Castor Oil or Polysorbate (20/60/80) or Tween (20/60/80) or any combination thereof.

20. The oral care composition of claim 16, wherein the sweetener is xylitol or sodium saccharin or stevia leaf extract or steviol glycosides or sucralose or erythritol or maltitol or mannitol or sorbitol or glycol and glycerol or aspartame or acesulfame K or any combination thereof.

21. The oral care composition of claim 16, wherein the rhamnolipid is derived from bacterium of a genus Burkholderia.

22. The composition of claim 21, wherein the rhamnolipid is derived from Pseudomonas spp.

23. The composition of claim 22, wherein the rhamnolipid is derived from Pseudomonas aeruginosa.

24. The composition of claim 22, wherein the rhamnolipid is derived from a rhamnolipid having the CAS number 4348-76-9.

25. The oral care composition of claim 16, wherein the composition is a mouthwash or a toothpaste.

26. The oral care composition of claim 16, wherein the oral care composition is in any of the following form: film, aerosol, suspension, solution, tincture, cream, paste, lotion, ointment, gel, powder, or granulate.

27. The oral care composition of claim 16, wherein plaque and/or biofilm being treated is derived from oral microorganisms in a mouth, wherein the oral microorganisms are selected from the group consisting of Streptococcus mutans, Streptococcus oralis, Streptococcus sanguinis, Neisseria mucosa and Actinomyces naeslundii.

28. The oral care composition of claim 16, wherein the composition is a medication formulated for application in a mouth of a subject.

29. The oral care composition of claim 16, wherein the composition is used for treatment of plaque and/or biofilm or for prevention of a plaque formation and/or biofilm formation in a mouth and/or on teeth.

30. The oral care composition of claim 29, wherein the plaque and/or the biofilm is on a solid surface of a tooth in a form of a supragingival plaque or a subgingival plaque.
